(19) United States
(12) Patent Application Publication
(10) Pub. No.: US 2022/0087227 A1
(43) Pub. Date: Mar. 24, 2022

(54) METHOD AND DEVICE FOR THE AUTOMATIC RECORDING OF THE MOVEMENT OF NEMATODES OR SMALL ORGANISMS OF SIMILAR SIZE BY THE TEMPORAL INTERFEROMETRY OF LIGHT MICROBEAMS

(71) Applicant: PHYLUMTECH S.A., Santa Fe (AR)

(72) Inventors: Sergio Hernan SIMONETTA, Alicante (ES); Mariano Javier SANTA CRUZ, Santa Fe (AR)

(21) Appl. No.: 17/423,813

(22) PCT Filed: Jan. 17, 2020
(86) PCT No.: PCT/ES2020/070029

§ 371 Date: Jul. 16, 2021

(30) Foreign Application Priority Data
Jan. 18, 2019 (AR) P20190100121

(51) Int. Cl. A01K 29/00; A01K 1/00; G01J 3/00; G01N 15/14; G06K 9/00; A61B 5/11; G01N 21/00

(52) U.S. Cl. CPC A01K 29/00; A01K 1/00; G01J 3/00; G01N 21/00; G06K 9/00; A61B 5/11; G01N 15/14

(57) ABSTRACT

Method and device for the automatic recording of the movement of nematodes or small organisms of similar size by the temporal interferometry of light microbeams. The method for the tracking of the locomotor activity of nematodes or small organisms of similar size, by means of the temporal interferometry of light microbeams, the device for the execution of the method, uses and applications of the method and device.

### TECHNOLOGICAL FIELD

The automated detection and quantification of the locomotor activity of tiny organisms has applications in the fields of discovery of new pharmacological, veterinary, agrochemical compounds, as well as nutraceuticals and studies of microbiological interactions. More specifically, its use has been reported in toxicity, stress resistance, search for new antibiotics, antiparasitic compounds, metabolism modulators and aging studies.

### BACKGROUND

The methods of detecting the behavior of small organisms using optical systems are mainly based on image capture and processing techniques.

There are various devices and procedures for recording the movement of small organisms and even cells such as sperm.

Chinese patent application CN107760757 discloses a device and procedure for recording nematodes movement in real time for the evaluation of drugs with antibacterial effect. The device comprises a light emitter, a porous culture plate, a plurality of cameras, a hollow plate arranged on the porous culture receptacle to protect light. A six-well plate is placed in the center of the upper structure, and the six-well plate contains the microporous insert, the culture medium, and the nematode. In order to record, quantify and determine the movements of nematodes the system needs to accurately and clearly detect the silhouette of each nematode, specifically using a high definition camera and image processing technique that recognize the contour, generating big size video files.

Chinese patent application CN103941752 discloses a real-time automatic tracking imaging system for nematodes comprising a light source device (providing bright field illumination), a four-axis moving device for objects in movement, an image collection device and a control device. The four-axis moving device for objects in movement, comprising a two-axis translation stage and a rotary table, is used to place a Petri dish containing a nematode and to adjust the position in the vertical plane of the vertical axis of the nematode. The nematode region of interest is always posicionated at the center of the collection area of the image capture device, according to instructions of the main control device.

U.S. Pat. No. 4,896,967 discloses a method and device for the study of sperm motility that comprises an imaging lens, a lighting source consisting of at least one LED (preferably between 3 and 12) with a single collimator aperture to focus light beam on the sample, and detection means of transmitted and scattered radiation, among others. The radiation emitted by LEDs is 880 nm infrared light.

U.S. Pat. No. 5,915,332 discloses a combined system to detect and to analyze animal activity. It combines the use of an IR light array with an ultrasonic phase shift system. The IR light array subsystem is used to record the horizontal position of an animal within a cage. Twenty-four pairs of IR light transmitters and receivers are attached to the cage wall in each direction (X, Y or Z axis). The horizontal movement and track of the animal are detected using infrared light, through analyzing how the infrared light is interrupted in the middle as the subject animal moves. The decoding circuit decodes the data from the IR light matrix subsystem and initiates an appropriate transmitter and receiver of the ultrasonic phase shift subsystem to detect the vertical change in behavior.

Patent application US2015204773 discloses a system for three-dimensional imaging of moving objects (specifically sperm) contained in a sample comprising: an image sensor; a sample holder configured to contain the sample, the sample holder disposed adjacent the image sensor; a first light source (red LED at 650 nm) with a first wavelength is placed in relation to the sample holder in a first location to illuminate the sample; a second light source (blue LED at 470 nm) with a second wavelength, different from the first wavelength, is positioned with respect to the sample holder at a second location, different from the first location. It also provides a method for three-dimensional tracking of moving objects by obtaining a plurality of image frames over time of the moving objects with an image sensor disposed adjacent to the sample holder. The image of the moving objects is digitally reconstructed based on the illumination originating from the first and second light sources, and then the x, y, and z positions of the moving objects are determined.

Gernat et al. (2018) developed a system to automatically monitor trophallaxis with high spatio-temporal resolution for long periods of time. The system allows the reliable identification and tracking of each individual in a colony from digital image sequences. Information about the position and orientation of each bee is used to identify the pairs of bees that were in the correct position to detect trophallaxis. The bees were housed in a glass-walled observation hive (a) that contained a single-sided honeycomb and was connected to a hole in the wall that allowed unlimited outside access to feed. The hive was illuminated with eight infrared LED lights mounted on an aluminum frame (b). To facilitate automatic image analysis, the honeycomb was backlighted with a series of infrared lights mounted behind the hive (c, hidden). The images were recorded with a high resolution monochrome camera (d) that controlled the infrared lights through a breakout panel.

Berh et al. (2017) show a Drosophila larval monitoring system. They use Frustrated Total Internal Reflection (FTIR) in combination with a multi-camera/microcomputer setup. To induce the FTIR effect, they have 12 IR LEDs with a dominant wavelength of 860 nm. When IR light enters the body of the semi-translucent animal, it is scattered through the larval tissue. The scattering process produces light rays with angles of incidence below the critical angle. These rays of light are no longer fully reflected. They are frustrated and can pass through the glass leading to the FTIR effect. This light is captured by the surrounding cameras. Therefore, the captured images show high contrast, as only the light scattered by the larvae is visible.

Perni et al. (2018) have developed a comprehensive nematode tracking platform (WF-NTP), which allows the simultaneous investigation of multiple phenotypic reads in large worm populations. The WF-NTP monitors up to 5000 animals in parallel, and the phenotypic reading includes multiple parameters. Combine a monochrome camera GS3-U3-60QS6M Grasshopper USB 3.0 1″ (Point Gray, Richmond, Calif.; 14-bit; 2736×2192 pixels) with a high-resolution 16 mm focal length f/1.8−f/16 lens to obtain a 6 to 14 cm image or a multi-well device with brightfield illumination (8″×8″ AI white side backlight) (Edmund Optics Ltd.).

Yu et al. (2014) disclose a simple and inexpensive multiwell technique to image up to 24 worms of any stage of development over several days. Individual worms are placed into small glass wells, allowing each animal to be tracked independently in high resolution. The imaging system consists of a camera, a lens, an LED illuminator, and mechanical components. The illuminator was a flexible red LED strip (48 cm long, 210 lumens, peak wavelength 619 nm).

Winbush et al. (2015) designed an image analysis tool to extract characteristics of movement (i.e., activity, trajectories, body posture) and shape (i.e., body length) from videos recorded over several days of multiple animals crawling on the surface of an agar plate. Using this population approach, measurements of the circadian locomotor activity of wild-type animals and temperature cycling-induced Caenorhabditis elegans mutants are obtained. The imaging setup consists of a digital camera, lens, red light LED illuminator, and mechanical parts. Animals on the test plate are illuminated using a low angle red light LED ring with an internal diameter of 110 mm and a light wavelength of 630 nm. This dark field illumination shows the animals as white objects on a relatively dark background.

The argentinian patent application AR058206 (A1) (from the same inventor of the present invention) discloses a small organism locomotor recording procedure and device, behavioral record obtained and use of the same. The procedure consists of impinging a microbeam of infrared light (λ=940 nm, with a power equal to or less than 1 mW/cm2) on the container (96-well microtiter plate, where there is 1 worm per well; extrapolated to plates of between 12 and 1536 wells) where the small organism (nematode) is found and to detect the scattering of light generated by the diffraction of the body of the organism. Each well must be irradiated by at least one microbeam. Subsequently, the signal is digitally processed to determine the locomotor activity of the organism. To generate the microbeams, the beam from at least one LED must be incident on a matrix with microholes. The array of microholes consists of an acrylic plate (2.5 mm wide) with 100 μm microholes aligned with the LEDs. The light scattered by the body of the organism when it passes through the microbeam is detected by phototransistors. Although this patent uses microbeams of light to detect the mobile microorganisms by refraction and has an adequate signal/noise index for liquid media with low absorbance in the infrared spectrum, its signal/noise index loses sensitivity when used with other more diffuse culture medium such as solids, semisolids or particulates, it is also not possible, by means of the device and method used, to be able to determine the spatial position of more than one organism per container. The method proposed here is superior in terms of the spatial resolution obtained, in addition to achieving a higher detection sensitivity (>1.5× compared to the original method) allowing a higher signal to noise with the background threshold and improving the detection of organisms, and consequently its locomotor activity, in semi-opaque culture media such as agar or bacteria cultures.

None of the cited documents mentions or suggests the use of a grid of adjacent parallel microbeams, nor does it mention the improvement in the signal/noise ratio generated by the effect of microbeam interferometry to detect the movement of organisms, nor is it possible to use with low resolution detection systems.

In this way, the present invention provides a method and device that uses such a configuration that allows an amplification effect of the signal/noise ratio for the detection of the movement of tiny organisms by means of a grid of adjacent parallel microbeams, capable of being used with low-resolution detection systems and real-time processing.

### BRIEF DESCRIPTION OF THE FIGURES

FIG. 1: Locomotive activity monitoring device diagram.

FIG. 3: Signal processing to obtain the quantification of locomotor activity. A: schematic block of the processing flow. B: graphical representation of signal processing.

FIG. 4: Continuous recording of the locomotor activity of 25 adult C. elegans cultured in solid culture plates (NGM) containing two different concentrations of the toxic Levamisole.

FIG. 5: Aging measurements and XY graphic location of the organisms: 20 adult worms were cultured in 35 mm plates for 10 days and recorded for 30 minutes, once a day.

### BRIEF DESCRIPTION OF THE INVENTION

The present invention describes a process to follow the locomotor activity of nematodes or small organisms of similar sizes that comprises the following steps:
a. having a plurality of small organisms that must be registered in a container comprising at least one receptacle where at least one organism is placed;
b. irradiating said container with a plurality of parallel light microbeams and adjacent to each other, separated by a distance of up to 1 mm, with the appropriate characteristics to produce a measurable interference with the body of the organism without altering the behavior or physiology of the organism, where said microbeams are generated by light previously filtered through a grid of microholes that precedes the container;
c. detecting the intensity or power of the microbeams crossing the container;
d. determine if there is fluctuation or attenuation in the intensity or power of the measured microbeams caused by the interference of the body of the organism when one of said microbeams passes through it, according to a detection threshold,
e. and recording the locomotor activity, including the shape and spatial location of the movement of the organism located within the container, based on the determination of fluctuations of the determined parameters of the microbeams.

Where the container contains a culture medium, with a low optical absorbance in the optical range of the wavelength of the microbeams. Preferably, said culture medium is selected from the set composed of: axenic, complex medium, with a source of live food, liquid, semi-solid, and solid.

In a preferred embodiment, said microbeams of light are infrared. In an another embodiment of the present invention, said microbeams of light are generated by at least one infrared LED lamp that emits infrared radiation, with a wavelength of between 700 to 1000 nm, preferably comprise a wavelength of between 850 to 950 nm, and said radiation is separated into microbeams by means of a grid of microholes. The power of these microbeams of light is less than 10 mW/cm2.

In another embodiment of the present invention, said microbeams are generated by a plurality of LEDs, each separated by a distance of between 1 to 5 mm.

In another way of carrying out the present invention, said microbeams are generated by a plurality of LEDs between 1 to 1000, each separated by a distance of 1 to 5 mm.

According to an another embodiment of the present invention, said grid of microholes comprises a thickness of at least 1 mm, and said microholes have a diameter of between 50 and 150 μm separated by a distance of up to 5 mm from each other, preferably said distance separation is up to 1 mm. More preferably, said separation distance is up to 3 mm.

Preferably, said grid of microholes can be a dark material, with low transmittance in the spectrum of the light source.

In another alternative embodiment of the present invention, prior to filtering the light, the microbeams of light are homogenized by means of a diffuser.

Furthermore, the present invention comprises a detection system that includes the conversion of the measured signal to an analog value directly proportional to the incident light. Also, a digital analog conversion system for incident light, where said conversion system comprises at least one camera with a resolution of at least 100×100 pixels.

In a preferred embodiment, the steps for detecting the fluctuation or attenuation in the intensity or power of the measured microbeams and of recording the locomotor activity of the organism include:
a. acquire at least two images at different times;
b. calculate the difference between both images for each pixel;
c. estimate for each one of the pixels if the absolute value of the differences is greater than a threshold value, where said threshold value is empirically determined as the maximum noise value obtained in plates that do not contain organisms, and increment a specific counter for each pixel in case the threshold is exceeded;
d. calculate the total activity by adding all the counters calculated in step (c).

The present invention further describes a device for measuring the locomotor activity of nematodes or small organisms of similar sizes, comprising:
a receptacle with at least one container suitable for cultivating said organisms;
a plurality of means generating infrared microbeams (1);
a grid of microholes of at least 1 mm thick, where said microholes have a diameter of between 50 and 150 μm separated by a distance of up to 5 mm from each other;
at least one infrared microbeam receiving means (4);
receiver circuit means for detecting variations in the output signal, and
a record linked to the output of the detector circuit means to register the locomotive activity, shape and position of the body's movement on the basis of the variations detected in the intensity or power of the microbeams.

Where the receptacle contains a medium with low infrared optical absorbance. And where, said microbeam generating means comprise LED light of wavelength between 850 to 950 nm, with an output power less than 10 mW/cm 2. Furthermore, the spacing of said microholes is preferably up to 1 mm; more preferably, the spacing of said microholes is up to 3 mm from each other.

In an alternative form, the device of the present invention further comprises a diffuser located between said microbeam generating means and said microhole grid.

In a preferred embodiment of the present invention, said receiving means comprises a digital analog converter. This digital analog converter comprises a photographic or video camera that can be low resolution. Furthermore, it comprises a processor circuit connected to the output of the digital analog converter and contains the algorithms for processing the acquired image signal.

In a preferred embodiment, the device of the present invention includes means to irradiate said habitats with daily light-dark cycles.

The present invention also describes a behavioral record obtained by the described procedure and the uses that can be: characterization of mutant organisms, toxicity of compounds, aging measurements and/or pharmacological tests.

### DETAILED DESCRIPTION OF THE INVENTION

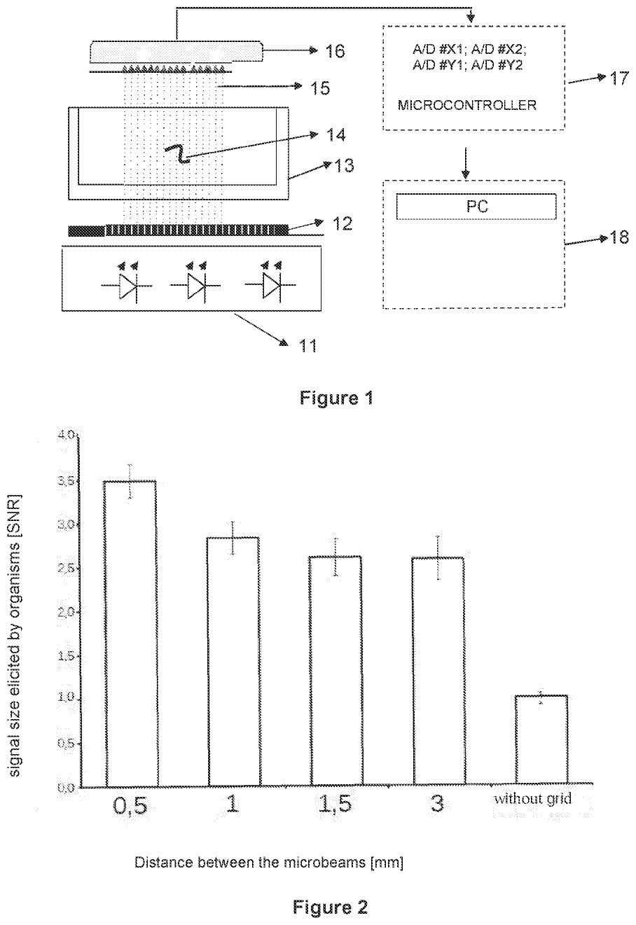

The object of the present invention comprises a method to follow the locomotor activity of nematodes or small organisms with a size between 10 μm and 20 mm. It also comprises a device of which it is possible to reproduce the method of the present invention that is represented in FIG. 1.

According to the method and device of the present invention it is possible to follow the locomotor activity of one or more small organisms simultaneously. The present invention allows knowing and following the pattern of movements, but also the shape and spatial location of the organism(s).

To carry out the method of the present invention, the small organisms must be placed in a container or surface. This container can be a 35 mm diameter culture plate, or it can also be culture microplates containing a plurality of receptacles, or smooth surfaces such as slides and plastic strips. In a preferred form, the container is selected from a set of: 35 mm diameter culture plate, 384, 96, 48, 12 and 6 wells microtiter plates, and even glass slides.

Said container must contain a culture medium that can be solid, semi-solid or liquid, axenic, complex or containing a live food source. Preferably with negligible absorbance (OD<1.0) in the light range on which the detection method is based.

Subsequently, the method comprises the irradiation of said receptacle containing said organisms with a plurality of microbeams of infrared light. These microbeams must be parallel and adjacent to each other, and also must be separated by a distance no greater than 1 mm from each other. This distance is essential in the method of the present invention because the sensitivity of the procedure for recording the locomotor activity of organisms depends on it. FIG. 2 shows the amplification effect of the signal/noise ratio obtained as a function of the distance between microbeams. It can be observed that the smaller the separation distance between the microbeams, the detection signal of the microorganisms significantly improves and therefore the procedure has a greater sensitivity.

Said microbeams can be generated by at least one infrared LED lamp, arranged below the container. These LED lamps' wavelength does not affect the organism. It is known in the state of the art that said wavelength varies between 700 and 1000 nm. Preferably between 800 and 950 nm. In an alternative embodiment, the infrared light generating source is an array of 10×10 LED lamps spaced 0.5-10 mm apart.

To generate said microbeams from said LED lamps, a grid of microholes must be employed where the LED light is applied. This grid of microholes is at least 1 mm thick, and comprises microholes of diameter between 50 and 150 μm separated by a distance of up to 5 mm from each other. In this way, the microbeams are generated due to the incidence of infrared light from said LED lamps on said grid of microholes. The thickness of the plate is important since when it receives the light, the microholes in the plate with said thickness will generate a tunnel effect that will make the beams coherent with each other.

In an alternative embodiment, said grid comprises a micro-hole spacing of between 0.1 and 3 mm. Preferably said the separation is between 0.5 and 1 mm.

In an alternative way of carrying out the method of the present invention, to homogenize the illumination of the container produced by the infrared microbeams, a diffuser can be used. This diffuser can be made of a 6 mm milky white acrylic plate placed just above the LEDs and before the plate with microholes.

After the microbeams impact and pass through said container containing the organisms and culture medium, the intensity of some of the microbeams changes due to their impact with the organism to be followed. This change in intensity, also called variation, interference or disturbance of the light beams, after passing through the body of the organism, is sensed by a receiving means, or a sensor, that comprises a digital analog conversion system of light. This system comprises a photo or video camera that can have a low resolution. An aspherical lens can be attached to said camera to improve the focus of the beams. Pixel intensity information is collected by an Arduino-Mega™ board-based acquisition system or equivalent, at an acquisition rate of 1 frame per second and transmitted at 1Mega Baud to a connected personal computer.

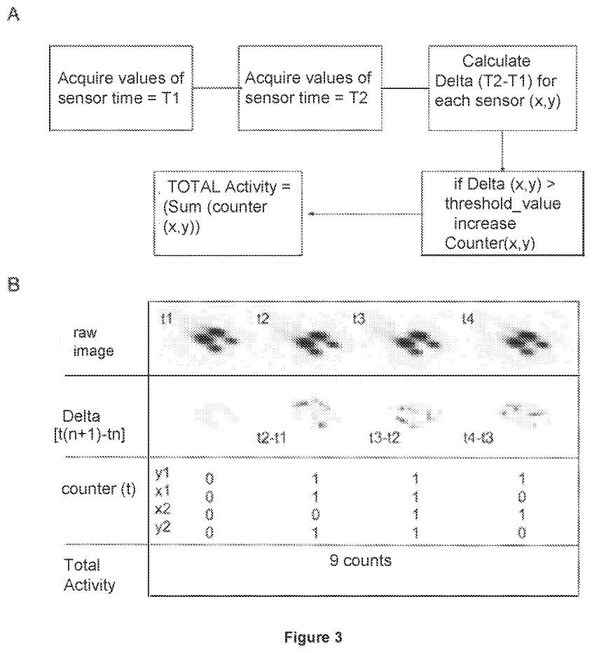

To be able to analyze the sensed fluctuations, one must proceed according to the scheme represented in FIG. 3. Briefly, at least two images must be acquired at different times; then the difference between the two times must be calculated for each pixel. Subsequently, it must be estimated for each of the pixels if the absolute value of the differences is greater than a threshold value, where said threshold value is empirically determined as the maximum noise value obtained in plates that do not contain organisms, and increase a specific counter for each pixel in case the threshold is exceeded; and finally, calculate the total activity by summation of all the counters.

In order to extract the movement of the organisms in the medium, a subtraction of consecutive image frames is carried out. The resulting image will contain the pixel difference value. In order to distinguish the organisms from the background noise of the acquisition it is necessary that the pixel difference value changes significantly with respect to the background noise signal (determined empirically as the threshold value), as illustrated in FIG. 3A.

When detecting a movement event in the container due to the variation of the intensity of the microbeams, an accumulator of global activity is increased, in addition to saving the data in an XY matrix that indicates the spatial places where activity was detected. Once the entire area has been scanned, the detected locomotive activity is reported, completing the calculation by integrating the activity. In this way, a dynamic calculation of the locomotor activity of the nematode population will be obtained over time with spatial information on their movement. This spatial information becomes relevant when doing population distribution studies in chemotaxis trials, social studies or models of diseases that affect movement patterns. In addition, it is a key piece of information in the application of algorithms that track individual organisms.

To carry out the procedure described above, is necessary a device (represented in FIG. 1) comprising:
An illumination source constituted by a plurality of infrared LED lamps (11) of power and wavelength as previously described;
A microperforated plate (grid of microholes) to generate the microbeams of parallel light and adjacent to each other (12);
Receptacle for housing organisms (13);
Small organism/s to monitor (14), which are traversed by the parallel microbeams of light (15) and whose movement is subsequently sensed;
Light receptor/s (sensor) (16) that captures the alterations in the microbeams that pass through the receptacle containing the organisms. This light receptors comprises an analog to digital converter that in turn can be constituted by a low resolution camera, for example, an ov7670 with 640×480 pixels, operating at a resolution of 140×140 pixels.
Receptor circuits (microcontroller) to capture, detect variations, and send (17) the measures to register means (18), which acquires the data and processes them according to a mathematical analysis algorithm to detect movement.

The descriptions below are examples of embodiments of the present invention which should be taken as such without limiting the scope of the invention.

### BEST MODE

### Example 1. Preferred Embodiment of the Invention

To carry out the experiment, the device was built according to FIG. 1. A diffusing plate was added to this model. The characteristics of each of the components of the device and each step of the method of the present invention are detailed below.

For the recording or monitoring of locomotor activity, the nematode C. elegans was used as a small organism. It was placed in a semi-solid culture medium called NGM (Nutrient Growth medium). This application is totally valid for organisms of the same order of size with the adjustment of the corresponding culture conditions.

In order to quantify the population behavior, 20 adult worms are housed in a container of a receptacle, such as a 35 mm Petri dish with 3 ml of NGM medium+monolayer of Escherichia coli bacteria (strain OP50) as food for the nematodes. The procedure is also valid for other container formats, such as 384, 96, 48, 24, 12 and 6-well microplates; with different solid, semi-solid and liquid culture media.

The container containing the nematodes is illuminated with a grid of infrared microbeams (monochromatic light wavelength between 850 and 950 nm) that does not affect the behavior of the animals, allowing a non-invasive measurement. Light emitters consisting of an arrangement of 10×10 OSRAM SFH 4356 LED lamps (850 nm) separated 4.5 mm from each other, and powered with a pulsating current of 10 mA 1 Khz have been used successfully. This configuration allows an infrared emission of 1 mW to 8 mW per LED. A better homogenization of the LED lighting was observed by using a diffuser made of a 6 mm milky white acrylic plate placed just above the LEDs and before the grid with microholes. The grid with microholes has been made by a laser cut pantograph system, containing microholes of 100 μm in diameter separated 0.5 mm from each other, made in a 2 mm thick black high impact plastic plate.

After passing through the culture medium, the beams are captured by a digital analog light conversion system. In the example case, a 640×480 pixel resolution CCD camera (ov7670 camera module, Omnivision® or similar) was used with a luminance data acquisition configuration at a QCIF frame resolution (176×144 pixels). An aspherical lens can be attached to said camera to improve the focus of the beams. Pixel intensity information is collected by an Arduino-Mega™ board-based acquisition system, at an acquisition rate of 1 frame per second and transmitted at 1 MB audio to an associated PC. The acquired signal is processed on an IBM-type personal computer by an ad hoc program made in Visual Basic (.NET), and Python capture systems can also be used on other platforms.

The processing of the captured image is carried out in real time according to the algorithm described in FIG. 3, which comprises the following steps:

i. the light intensity values of the image are acquired through serial communication with the ARDUINO microcontroller and stored in a memory array, indexed with their corresponding XY spatial position pair;
ii. from the individual intensity values, the value of the previous image (n−1) is subtracted and the difference is saved in another vector called delta_image (x, y);
iii. a sweep loop is made for the delta_image vector (x, y) comparing the data with a threshold_value. If for each point, the absolute value of the delta_image vector (x, y) is greater than the threshold_value, then a specific counter is incremented for said spatial position of the image: counter (x, y)=counter (x, y)+1;
iv. at the end of the sweep loop, all the counters (x, y) are summed and stored in another array Total_Activity (t) that has a time index attached;
v. the previous steps are repeated until the end of the acquisition period desired by the user, which can vary from 1 minute to several days;
vi. at the end of the acquisition period, the total_activity (t) with its corresponding time value is reported in a table. With this information, the user will be able to plot the activity kinetics of the organisms in an activity vs. time graph, or they will be able to integrate these values to obtain the global activity in the determined period.

### Example 2. Application of the Registry for the Measurement of the Toxicity Effect of Compounds

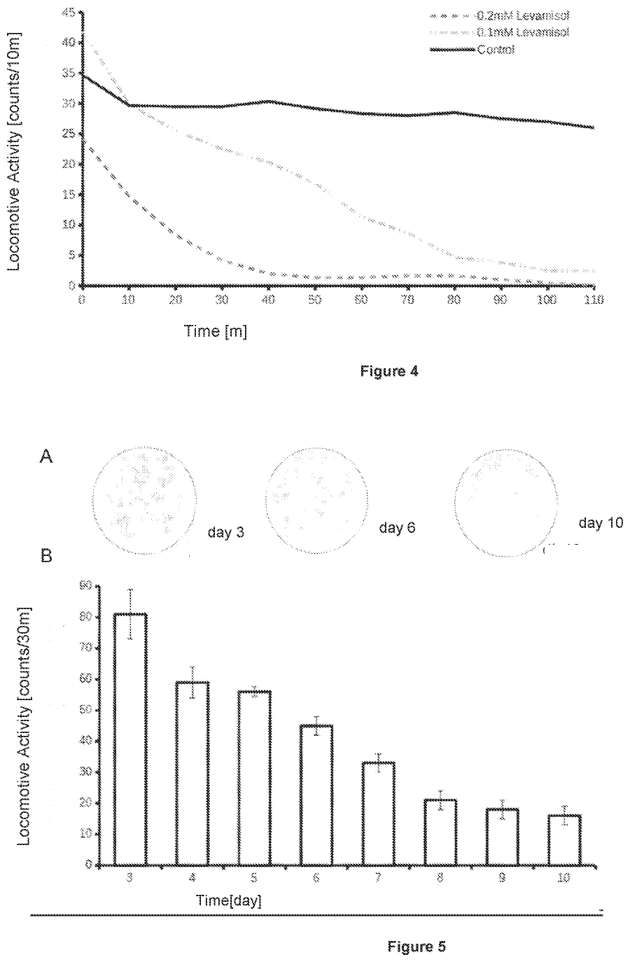

The experiment was carried out according to example 1 with the necessary modifications to evaluate the toxicity of certain drugs in nematodes. As a container, 35 mm Petri dishes with NGM were used containing concentrations of Levamisole (antiparasitic with known effect) at concentrations of 0 mM, 0.1 mM and 0.2 mM in duplicate. After adding 25 worms to each plate, locomotor activity was measured for 2 hours. Cumulative activity was then plotted in 10 minute blocks for all plates. FIG. 4 shows a continuous record of the locomotor activity of 25 adult C. elegans grown in solid culture plates (NGM) for 2 conditions of the toxic levamisole. Each point consists in the average of experimental duplicates, grouping the activity in blocks of 10 minutes. It can be seen that the controls (untreated plates) show a constant activity over time, while 0.1 mM and 0.2 mM rapidly decay in a dose-dependent manner.

### Example 3. Application of the Registry for the Measurement of the Aging of Nematodes

The C. elegans model was used as an experimental animal due to its high aging rate. For the experiment, 35 mm Petri dishes with NGM with 100 uM FuDr (Fluorodeoxyuridine, a nematode reproduction inhibitor)+E. coli (OP50) were used in sixtuplicates. 20 larva worms 4 (L4) were added to each plate and the locomotor activity of each plate was measured once a day, 1 hour, for 10 consecutive days from day 3 of adult; adding an additional drop of OP50 every 6 days to avoid worm starvation. In FIG. 5 it can be seen that the device and procedure are capable of detecting the decay of locomotor activity with the age of the worms, with relevance in the field of discovery of new drugs and genes involved in the delay of aging and/or senescence. FIG. 5A shows the XY capture points of the experimental plates where movement has been detected at several temporal measurements for 3 different days. The black spots within the circle correspond to pixels that have exceeded the detection threshold in the 30 minute period. It can be seen that a decline in nematode population activity with aging is detectable by this method. In FIG. 5B the bar graph shows the quantification of the locomotor activity (experimental duplicates) and its decay with nematode aging.

1-32. (canceled)

33. A method for the automatic recording of the locomotion of nematodes or small organisms of similar sizes by temporal interferometry of light microbeams, comprising the following stages:
providing a plurality of small organisms that must be registered in a container comprising at least one receptacle where at least one organism is placed;
irradiating the container with a plurality of microbeams of light parallel and adjacent to each other, separated by a distance of up to 4 mm, with the appropriate characteristics to produce a measurable interference with the body of the organism without altering the behavior or physiology of the organism, where the microbeams are generated by light previously filtered through a grid of microholes that precedes the container;
detecting the intensity or power of the microbeams crossing the container;
determining if there is fluctuation or attenuation in the intensity or power of the measured microbeams caused by the interference organism body when one of the microbeams passes through it, according to a detection threshold; and
recording the locomotor activity, including the shape and spatial location of the movement of the organism located within the container, based on the determination of fluctuations of the determined parameters of the microbeams.

34. The method of claim 33, wherein the container comprises a culture medium, with low absorbance in the optical range of the wavelength of the microbeams.

35. The method of claim 33, wherein the culture medium is selected from the set consisting of axenic, complex, live food, liquid, semi-solid, and solid food source.

36. The method of claim 33, wherein the microbeams are infrared light of a wavelength between 700 to 1000 nm and a power less than 10 mW/cm².

37. The method of claim 33, wherein the microbeams of light are generated by at least one LED lamp that emits infrared radiation and the radiation is split into microbeams by means of a grid of microholes.

38. The method according to claim 33 wherein the microbeams are generated by a plurality of LEDs, each separated by a distance range from 1 to 5 mm.

39. The method of claim 33, wherein the microholes grid plate comprise a thickness of at least 1 mm, and wherein the microholes comprise a diameter between 50 and 150 μm separated by a distance of up to 5 mm from each other.

40. The method of claim 33, wherein the grid of microholes is made of a dark material, with low transmittance in the spectrum of the light source.

41. The method of claim 33, wherein prior to filtering, the microbeams of light are homogenized by means of a diffuser.

42. The method of claim 33, wherein it comprises a detection system that includes the conversion of the measured signal to an analog value directly proportional to the incident light, an analog to digital conversion system for incident light, and the conversion system comprises at least one camera with a resolution of at least 100×100 pixels.

43. The method of claim 33, wherein the steps of detecting the fluctuation or attenuation in the intensity or power of the measured microbeams and recording the locomotor activity of the organism include:
a. acquiring at least two images at different times;
b. calculating the difference between both times for each pixel;
c. estimating for each one of the pixels if the absolute value of the differences is greater than a threshold value, where the threshold value is empirically determined as the maximum noise value obtained in plates that do not contain organisms; incrementing a specific counter for each pixel in case the threshold is exceeded;
d. calculating the total activity by summing all the counters calculated in step (c).

44. A device for measuring the locomotor activity of nematodes or small organisms of similar sizes, comprising:
a container with at least one receptacle suitable for cultivating said organisms;
a plurality of means generating infrared microbeams;
a plate grid of microholes of at least 1 mm thick, where the microholes have a diameter of between 50 and 150 μm separated by a distance of up to 5 mm from each other;
at least one infrared microbeam receiving means;
receiver circuit means for detecting variations in the output signal; and
a register linked to the output of the receiver circuit means to record the locomotor activity, shape and position of the body's movement on the basis of the variations detected in the intensity or power of the microbeams.

45. The device according to claim 44, wherein the receptacle contains a medium with low optical absorbance in the infrared spectrum and the microbeam generating means comprise LED lamps of wavelength between 850 and 950 nm, an output power less than 10 mW/cm².

46. The device of claim 44, wherein the microholes of the grid are separated by a distance of up to 4 mm from each other.

47. The device of claim 44, wherein it further comprises a diffuser located between the micro-beam generating means and the micro-hole grid.

48. The device of claim 44, wherein the infrared beam receiver means comprises an analog to digital converter, a photographic or video camera of low resolution and a processor circuit connected to the output of the receiving means and containing processing algorithms acquired image signal.

49. The device of claim 44, further comprising means for irradiating the habitats with daily light-dark cycles.
